(19) United States
(12) Patent Application Publication
(10) Pub. No.: US 2006/0287394 A1
(43) Pub. Date: Dec. 21, 2006

(54) Composition and method for treating macular disorders

(76) Inventor: Konstantin Petrukhin, Collegeville, PA (US)

Correspondence Address: MERCK AND CO., INC, P O BOX 2000, RAHWAY, NJ, 07065-0907, US

(21) Appl. No.: 10/557,170

(22) PCT Filed: May 26, 2004
(86) PCT No.: PCT/US04/16513

§ 371 Date: Nov. 17, 2005

Related U.S. Application Data
(60) Provisional application No. 60/474,776, filed on May 30, 2003.

(51) Int. Cl. A61K 31/18

(52) U.S. Cl. 514/602

(57) ABSTRACT

A method and composition for treating a macular condition selected from the group consisting of macular degeneration or macular edema. A therapeutically effective amount of a carbonic anhydrase isoform IX inhibitor is administered to the patient to normalize intracellular pH of retinal pigment epithelial cells. The carbonic anhydrase isoform IX inhibitor can be administered alone or in combination with a therapeutically effective amount of an ocular hypotensive agent sufficient to improve visual function.

### BACKGROUND OF THE INVENTION

Macular degeneration results from accumulations of lipofuscin, a metabolic waste product in the cells of the retinal pigment epithelium (RPE). Each RPE cell apposes approximately 40 photoreceptor outer segmnets; 10-15% of outer segment length is phagosytosed daily by RPE. The lipofuscin is believed to accumulate as a result of incomplete lysosomal digestion of the photoreceptor outer segments phagocytosed by RPE cells. Shedding (phagocytosis) of photoreceptor outer segments is constantly occurring in a healthy retina. Good retinal pigment epithelial metabolism generally ensures a rapid lysosomal degradation and clearance of such catabolic by-products of vision. RPE cell lysosomes contain many pH-sensitive lytic enzymes (cathepsin D, cathepsin B, alpha-mannosidase, etc.) important for degradation and clearance of photoreceptor outer segments. Abnormalities in regulation of intracellular pH in RPE cells reduce the activity of pH-sensitive lysosomal enzymes and increase the process of lipofuscinogenesis. The accumulation of lipofiscin retards the interaction between the neuroretina and the retinal pigment epithelium from which nutrients arrive and through which catabolites are cleansed establishing a vicious cycle of catabolite accumulation. Secondarily to excessive accumulation of lipofuscin within RPE cells drusen accumulate below the RPE layer in number and begin to coalesce, vast areas of retinal photoreceptors may become permanently disengaged from their neighboring retinal pigment epithelial villi. The sections of retina so affected become blind. The greatest propensity among the aging population is for drusen to accumulate in the very central area of vision, the macula. Macular degeneration is the most common cause of legal blindness in the United States and Europe. Acetazolamide, a carbonic anhydrase inhibitor, has been given orally to treat macular edema but, while helpful, produces unpredictable responses and characteristically generates many systemic side effects. Even with the lower doses used in treatment of macular edema, a large proportion of patients fail to continue therapy because of poor drug tolerance.

Macular edema is swelling within the retina in the critically important central visual zone at the posterior pole of the eye. Wolfensberger, T. J. Invest. Ophthalmol. Vis. Sci. 1999; 97 (34); 387-97 describes the role of carbonic anhydrase inhibitors in the management of macular edema. An accumulation of fluid tends to distract the retinal neural elements from one another and from their local blood supply, creating a dormancy of visual function in the area. Usually, the process is self-limiting, but occasionally permanent visual disability results from macular edema. Often times, the swelling may take many months to clear. The precise mechanism by which swelling is triggered is uncertain, but it is probable that certain natural metabolic toxins may play an important role in the disease process. Macular swelling may also follow the insertion of artificial lens implants and cataract surgery, particularly if there is a breach in the lens capsule which segregates the vitreous gel from the fluid-filled anterior chamber. Longstanding macular edema after cataract surgery is one of the most frustrating dilemmas in all of ophthalmology, and is remarkably common.

Two types of cystoid macular edema are those without vascular leakage (retinitis pigmentosa and other pigmentary retinal degenerative disorders, early stage macular hole, and choridal neovascularization) and those with vascular leakage (diabetic retinopathy; branch retinal vein occlusion; intermediate uveitis; and ideopathicretinaltelangiectasis).

Whereas macular edema typically affects only one eye, macular degeneration typically involves both eyes and is usually fairly symmetric in its presentation and progression.

Wolfensberger, T. J. et al., Invest. Ophthalmol. Vis. Sci. August 1994; 35 (9); 3401-7 describes membrane-bound carbonic anhydrase in human retinal pigment epithelium. The paper indicates that carbonic anhydrase isoform IV is important for in the treatment of macular edema. However, isoform IV is not present in the retinal pigment epithelium, and it is inhibition of carbonic anhydrase isoform IX that is effective in treating macular degeration and macular edema.

The invention relates to a composition and method for treating different inherited and sporadic forms of macular and retinal degeneration and macular and retinal edema through the application of a topical carbonic anhydrase isoform IX inhibitor and, optionally, an ocular hypotensive agent or inotropic agents in an amount sufficient to improve visual function. Other retinal disorders that can be treated are retinitis pigmentosa, familial drusen, and macular disorders related to hypertension, angioma, papillitis, neuro retinitis (including Lebers stellate retinopathy) and other pigmentary retinal degenerative disorders. Carbonic anhydrase isoform IX inhibitors normalize intracellular pH of retinal pigment epithelial cells, and decrease the rate of lipofuscinogenesis.

### SUMMARY OF THE INVENTION

The present invention comprises compositions and methods for treating a macular condition selected from the group consisting of macular degeneration (including age-related macular degeneration and inherited forms of macular degeneration such as Best's disease and Stargardt's macular dystrophy) and macular edema due to retinitis pigmentosa or diabetic retinopathy, by administering a therapeutically effective amount of a carbonic anhydrase isoform IX inhibitor, either to the eye or systemically. Preferably, the invention involves topical application of the carbonic anhydrase isoform IX inhibitor in an amount effective to ameliorate the macular degeneration or macular edema. The instant invention provides an effective treatment for maintaining the health of the eye and effectively treating macular degeneration or macular edema.

The invention includes compositions for treating macular degeneration or macular edema comprising a therapeutically effective amount of a carbonic anhydrase isoform IX inhibitor and a pharmaceutically acceptable carrier. Compositions of the invention include ophthalmic preparation such as solutions, gels, semisolids, suspensions, metered dose devices, transdermal patches and films. In one embodiment, the ophthalmic preparation comprises a carbonic anhydrase isoform IX inhibitor at a concentration of about 0.01% weight/volume to about 5% weight/volume.

The invention is also a method for treating a macular condition selected from the group consisting of macular degeneration and macular edema in a patient, by administering to the patient a therapeutically effective amount of a carbonic anhydrase isoform IX inhibitor. In one embodiment, the macular condition results from retinitis pigmentosa or diabetic retinopathy. In another embodiment, the macular degeneration is age-related macular degeneration or an inherited form of macular degeneration. In another embodiment, the inherited form of macular degeneration is selected from the group consisting of Best's disease and Stargardt's macular dystrophy.

### DETAILED DESCRIPTION OF THE INVENTION

Carbonic anhydrase isoform IX inhibitors normalize intracellular pH within the cells of retinal pigment epithelium and decrease the rate of lipofuscinogenesis. Accumulation of lipofuscin in the retinal pigment epithelium is a significant risk factor for age-related macular degeneration, as can be judged by increased accumulation of lipofuscin in the junctional zone of geographic atrophy. Abnormal accumulation of lipofuscin is the cause of at least two inherited forms of macular degeneration, Stargardt macular dystrophy and Best's disease. Reduction of the rate of lipofuscin accumulation in the retinal pigment epithelium is an effective treatment for age-related macular degeneration. It is also effective for treating inherited forms of macular dystrophy.

Lipofuscin accumulates in retinal pigment epithelium cells because of incomplete digestion of shed outer segments of photoreceptor cells in retinal pigment epithelium phagolysosomes. Incomplete lysosomal degradation is caused by reduced activity of pH-sensitive lysosomal enzymes and/or reduced activity of lysosomal iron transporter Nramp2 that removes iron, an established pro-lipofuscinogenic agent, from lysosomes using the proton gradient as a driving force. Abnormal pH regulation in retinal pigment epithelium (alkalinization of the cytoplasm) would diminish the proton gradient across the lysosomal membrane, change pH optima for lysosomal enzymes, and increase the concentration of highly pro-oxidant iron within the phagolysosomes. This would lead to an increased rate of lipofuscinogenesis in retinal pigment epithelium cells. Abnormal pH regulation is involved in pathogenesis of macular degeneration. Evidence of Best's macular dystrophy are increased accumulation of lipofuscin and diminished light peak to dark trough ratio on electrooculogram. Electrooculogram is mediated by pH-sensitive chloride channels located on the basolateral side of retinal pigment epithelium cells. Bestrophin appears to represent a component of the pH-regulating system in the retinal pigment epithelium cells. Deficiency in pH regulation would explain both phenotypes characteristic of Best disease (lipofuscin and abnormal electrooculogram).

Abnormal pH regulation appears to be involved in the pathogenesis of macular degeneration. Carbonic anhydrase isoforms represent an important component of pH regulation in the cell. Activation of carbonic anhydrases would result in alkalinization of the cytoplasm and, potentially, lysosomes. There is a group of recently discovered membrane-bound carbonic anhydrase isoforms (carbonic anhydrase isoform IX and carbonic anhydrase isoform XII) which were originally found in cancer cell lines containing inactivated von Hippel-Lindau tumor suppressor gene. Most of the normal adult human tissues, as well as cancer cell lines with active von Hippel-Lindau, do not express carbonic anhydrase isoform IX and carbonic anhydrase isoform XII. The expression of both genes is markedly induced under hypoxic conditions. There is a pronounced expression of carbonic anhydrase isoform IX in the cells of retinal pigment epithelium and no detectable levels of carbonic anhydrase isoform XII, carbonic anhydrase isoform II or carbonic anhydrase isoform IV. Activation of carbonic anhydrase isoform IX by relative hypoxia would increase alkalinization of retinal pigment epithelium cells, reduce the lysosomal degradation of shed photoreceptor outer segments leading to a subsequent increase in the rate of lipofuscinogenesis. Carbonic anhydrase inhibitors decrease intracellular pH. Inhibition of carbonic anhydrase isoform IX reduces the rate of lipofuscinogenesis in retinal pigment epithelium, and specific carbonic anhydrase isoform IX inhibitors are useful for the treatment, and prevention, of macular degeneration and macular edema.

In accordance with the present invention, carbonic anhydrase isoform IX inhibition specific for the isoform expressed in the retinal pigment epithelium, would inhibit carbonic anhydrase isoform IX, providing the described therapeutic benefits with minimal systemic side effects. Several carbonic anhydrase inhibitors have been shown to cause systemic acidosis, caused by inhibition of carbonic anhydrase isoform IV in the kidney. Inhibitors of carbonic anhydrase isoform II would also cause side effects since carbonic anhydrase isoform II is a widely expressed isoform.

Carbonic Anhydrase Isoform IX Inhibitors

Isoform-specific carbonic anhydrase inhibitors are identified by comparing their ability to inhibit various carbonic anhydrase isoforms. For example, dorzolamide, a carbonic anhydrase isoform II-specific inhibitor when compared with its potential to inhibit carbonic anhydrase isoform IV and carbonic anhydrase isoform I, was identified as such by Sugrue M. F., J. Ocular Pharmacology, 12, 363-376, 1996.

Carbonic anhydrase isoform IX inhibitors suitable for use in the present invention are those which display selectivity for inhibiting carbonic anhydrase isoform IX over other carbonic anhydrase enzymes, e.g. at least 100 times more selective for carbonic anhydrase isoform IX compared to carbonic anhydrase isoform II or carbonic anhydrase isoform IV.

A panel of recombinantly expressed carbonic anhydrase isoforms is generated by expression in E. coli using conventional expression techniques (for carbonic anhydrase IX, see Pastorekova et al. Gastroenterology 1997:112:398-408; Ivanov et al. Proc. Natl. Acad Sci USA 95 pp. 12596-12601; and Opavsky et al. Genomics 33, 480-487 (1996)). The panel is used in a uniform standard assay for identifying isoform inhibitors. Carbonic anhydrase isoform IX specific inhibitors are determined by measuring the inhibition of each isoform obtained by a test compound, and comparing the measurements.

Carbonic anhydrase isoforms are purified to homogeneity as required to determine selectivity. Carbonic anhydrase isoform I and carbonic anhydrase isoform II are obtained in pure form from red blood cells, and carbonic anhydrase isoform IV from lung (Sugrue M. F., J. Ocular Pharmacology, 12, 363-376, 1996). Carbonic anhydrase isoform IX is purified from renal cell carcinomas overexpressing this isoform (PNAS, 95, 7608-7613, 1998). Carbonic anhydrase isoforms IX, XII and XIV are purified after their cDNA is expressed in any of the known heterogeneous expression systems (mammalian, bacterial, or insect). Such systems are well known and widely used. For purification, a carbonic anhydrase isoform is tagged with a widely used sequence (six histidines, GST, maltose-binding protein, myc, flag) so the well established purification method (Ni columns for six histidines, GH-Sepharose for GST, antibody columns for myc and flag) can be used to purify the protein to homogeneity. Enzymatic activity of all carbonic anhydrase isoforms can be measured using established and widely used techniques. One of such techniques is a so-called Hansson technique in which cobalt-sulfide precipitate is formed as a result of enzyme activity (Hansson H P J, Histochemie. 1967, 11, 112-128). Another established technique for measuring carbonic anhydrase activity is based on measuring changes in pH as a result of carbonic anhydrase activity (Maren T H J. Pharmacol. Exp. Ther. 130, 26-29, 1960).

The method is based on the rapid pH change due to carbonic anhydrase activity in the test solution. The test tube (containing phenol red (indicator color) in distilled water and purified carbonic anhydrase IX) is placed next to a pH standard tube (phenol red+water+phosphate, pH 7.2). CO₂ is continuously bubbled to saturate the test solution. After 60 seconds of uninterrupted CO₂ flow, diluted enzyme and thereafter barbital buffer, pH 7.9, are added, and timing by a stopwatch is begun. The reaction reaches the end point when the indicator color matches that of the 7.2 pH standard. Uncatalyzed time represents the time required for spontaneous return of the test solution to acidity due to the uncatalyzed hydration of CO₂ when only water instead of enzyme is added prior to the association of the barbital buffer. The carbonic anhydrase activity unit was calculated as follows: (uncatalyzed time-catalyzed time)/catalyzed time. Specific activity was represented as carbonic anhydrase activity units/mg of purified protein.

Carbonic anhydrase inhibitors are tested for their ability to inhibit carbonic anhydrase isoform IX in vivo using an assay based on measurements of fluid absorption across retinal pigment epithelium in rabbit model of retinal detachment (Wolfensberger T. J., Graefes Arch Clin Exp Ophthalmol. January 2000; 238(1); 76-80).

“Pharmaceutically acceptable salts” means non-toxic salts of the compounds employed in this invention which are generally prepared by reacting the free acid with a suitable organic or inorganic base. Examples of salt forms of carbonic anhydrase isoform IX inhibitors may include, but are not limited to, acetate, benzenesulfonate, benzoate, bicarbonate, bisulfate, bitartrate, borate, bromide, calcium, calcium edetate, camsylate, carbonate, chloride, clavulanate, citrate, dihydrochloride, edetate, edisylate, estolate, esylate, fumarate, gluceptate, gluconate, glutamate, hexylresorcinate, hydrabamine, hydrobromide, hydrochloride, hydroxynapthoate, isothionate, lactate, lactobionate, laurate, malate, maleate, mandelate, mesylate, methylsulfate, mucate, oleate, oxalate, pamaote, palmitate, panthothenate, phosphate/diphosphate, polygalacturonate, potassium, salicylate, sodium, stearate, subacetate, succinate, tannate, tartrate, teoclate, tosylate, and valerate.

Unless defined otherwise, “therapeutically effective amount” means that amount of a drug or pharmaceutical agent that will elicit the biological or medical response of a tissue, a system, animal or human that is being sought by a researcher, veterinarian, medical doctor or other clinician.

Unless defined otherwise, “prophylactically effective amount” means that amount of a pharmaceutical drug that will prevent or reduce the risk of occurrence of the biological or medical event that is sought to be prevented in a tissue, a system, animal or human by a researcher, veterinarian, medical doctor or other clinician.

The term “patient” includes mammals, especially humans, who take a carbonic anhydrase isoform IX inhibitor for any of the uses described herein.

Administration

The carbonic anhydrase isoform IX inhibitors of the invention can be administered in such oral forms as tablets, capsules (each of which includes sustained release or timed release formulations), pills, powders, granules, elixers, tinctures, suspensions, syrups, and emulsions. Likewise, they may be administered in intravenous (bolus or infusion), intraperitoneal, subcutaneous, or intramuscular form, all using forms well known to those of ordinary skill in the pharmaceutical arts.

The carbonic anhydrase isoform IX inhibitors can be administered in the form of a depot injection or implant preparation which may be formulated in such a manner as to permit a sustained release of the active ingredient. The active ingredient can be compressed into pellets or small cylinders and implanted subcutaneously or intramuscularly as depot injections or implants. Implants may employ inert materials such as biodegradable polymers or synthetic silicones, for example, Silastic, silicone rubber or other polymers manufactured by the Dow-Corning Corporation.

The carbonic anhydrase isoform IX inhibitors can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The carbonic anhydrase isoform IX inhibitors may also be delivered by the use of monoclonal antibodies as individual carriers to which the compound molecules are coupled. The carbonic anhydrase isoform IX inhibitors may also be coupled with soluble polymers as targetable drug carriers. Such polymers can include polyvinlypyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the thrombin inhibitors may be coupled to a class of biodegradable polymers useful in achieving controlled release of a drug, for example; polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

The dosage regimen utilizing the carbonic anhydrase isoform IX inhibitors is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Oral dosages of the carbonic anhydrase isoform I inhibitors, when used for the indicated effects, will range between about 0.01 mg per kg of body weight per day (mg/kg/day) to about 30 mg/kg/day, preferably 0.025-7.5 mg/kg/day, more preferably 0.1-2.5 mg/kg/day, and most preferably 0.1-0.5 mg/kg/day (unless specificed otherwise, amounts of active ingredients are on free base basis). For example, an 80 kg patient would receive between about 0.8 mg/day and 2.4 g/day, preferably 2-600 mg/day, more preferably 8-200 mg/day, and most preferably 8-40 mg/kg/day. A suitably prepared medicament for once a day administration would thus contain between 0.8 mg and 2.4 g, preferably between 2 mg and 600 mg, more preferably between 8 mg and 200 mg, and most preferably 8 mg and 40 mg, e.g., 8 mg, 10 mg, 20 mg and 40 mg. Advantageously, the carbonic anhydrase isoform IX inhibitors may be administered in divided doses of two, three, or four times daily. For administration twice a day, a suitably prepared medicament would contain between 0.4 mg and 4 g, preferably between 1 mg and 300 mg, more preferably between 4 mg and 100 mg, and most preferably 4 mg and 20 mg, e.g., 4 mg, 5 mg, 10 mg and 20 mg.

Intravenously, the patient would receive the active ingredient in quantities sufficient to deliver between 0.025-7.5 mg/kg/day, preferably 0.1-2.5 mg/kg/day, and more preferably 0.1-0.5 mg/kg/day. Such quantities may be administered in a number of suitable ways, e.g. large volumes of low concentrations of active ingredient during one extended period of time or several times a day, low volumes of high concentrations of active ingredient during a short period of time, e.g. once a day. Typically, a conventional intravenous formulation may be prepared which contains a concentration of active ingredient of between about 0.01-1.0 mg/ml, e.g. 0.1 mg/ml, 0.3 mg/ml, and 0.6 mg/ml, and administered in amounts per day of between 0.01 ml/kg patient weight and 10.0 ml/kg patient weight, e.g. 0.1 ml/kg, 0.2 ml/kg, 0.5 ml/kg. In one example, an 80 kg patient, receiving 8 ml twice a day of an intravenous formulation having a concentration of active ingredient of 0.5 mg/ml, receives 8 mg of active ingredient per day. Glucuronic acid, L-lactic acid, acetic acid, citric acid or any pharmaceutically acceptable acid/conjugate base with reasonable buffering capacity in the pH range acceptable for intravenous administration may be used as buffers. The choice of appropriate buffer and pH of a formulation, depending on solubility of the drug to be administered, is readily made by a person having ordinary skill in the art.

The compounds can also be administered in intranasal form via topical use of suitable intranasal vehicles, or via transdermal routes, using those forms of transdermal skin patches well known to those of ordinary skill in that art. To be administered in the form of a transdermal delivery system, the dosage administration will, or course, be continuous rather than intermittent throughout the dosage regime.

The carbonic anhydrase isoform IX inhibitor may preferably be administered as a 0.01-5%, preferably a 0.5 to 2% solution or suspension, in an ophthalmologically acceptable carrier.

The carbonic anhydrase isoform DC inhibitors are typically administered as active ingredients in admixture with suitable pharmaceutical diluents, excipients or carriers (collectively referred to herein as “carrier” materials) suitably selected with respect to the intended form of administration, that is, oral tablets, capsules, elixers, syrups and the like, and consistent with convention pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water and the like. Moreover, when desired or necessary, suitable binders, lubricants, distintegrating agents and coloring agents can also be incorporated into the mixture. Suitable binders include starch, gelatin, natural sugars such as glucose or beta-lactose, corn-sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch methyl cellulose, agar, bentonite, xanthan gum and the like.

Combination

The instant invention also provides pharmaceutical compositions comprised of a therapeutically effective amount of a carbonic anhydrase isoform IX inhibitor, or a pharmaceutically acceptable salt thereof, in combination with a therapeutically effective amount of an ocular hypotensive agent, or a pharmaceutically acceptable salt thereof, and a pharmaceutically acceptable carrier.

Ocular hypotensive agents including, but not limited to, beta blockers (betaxolol, timolol, optipranolol, levobunolol, metapranolol, carteolol, and the like), miotic agents (pilocarpine, carbachol, phospholine iodide, and the like), adrenergic agonists (ilopidine, brimonidine, epinephrine, dipivephrin, and the like), prostaglandin derivatives (latanoprost and the like), may be included in compositions of the invention. Additionally, compounds directed toward the reduction of intraocular pressure, plus agents effective in the enhancement of carotid perfusion pressure, including a range of oral and sublingual systemic drugs intended to improve cardiac contractility or decrease carotid or ophthalmic arterial vascular resistance, may be included in compositions of the invention.

In the following formulations, Active I is a selective carbonic anhydrase isoform IX inhibitor identified according to the procedure described above.

### EXAMPLE 1

Tablet Preparation

Tablets containing 25.0, 50.0, and 100.0 mg., respectively, of the following active compounds are prepared as illustrated below (compositions A-C).

| | Amount-(mg) | | |
|---|---|---|---|
| Component | A | B | C |
| Active I | 25 | 50 | 100 |
| Microcrystalline cellulose | 37.25 | 100 | 200 |
| Modified food corn starch | 37.25 | 4.25 | 8.5 |
| Magnesium stearate | 0.5 | 0.75 | 1.5 |

All of the active compound, cellulose, and a portion of the corn starch are mixed and granulated to 10% corn starch paste. The resulting granulation is sieved, dried and blended with the remainder of the corn starch and the magnesium stearate. The resulting granulation is then compressed into tablets containing 25.0, 50.0, and 100.0 mg, respectively, of active ingredient per tablet.

Tablet Preparation via Direct Compression

Active I, mannitol and microcrystalline cellulose are sieved through mesh screens of specified size (generally 250 to 750 μm) and combined in a suitable blender. The mixture is subsequently blended (typically 15 to 30 min) until the drug was uniformly distributed in the resulting dry powder blend. Magnesium stearate is screened and added to the blender, after which a precompression tablet blend is achieved upon additional mixing (typically 2 to 10 min). The precompression tablet blend is then compacted under an applied force, typically ranging from 0.5 to 2.5 metric tons, sufficient to yield tablets of suitable physical strength with acceptable disintegration times (specifications will vary with the size and potency of the compressed tablet). In the case of the 2, 10 and 50 mg potencies, the tablets are dedusted and film-coated with an aqueous dispersion of water-soluble polymers and pigment.

Tablet Preparation via Dry Granulation

Alternatively, a dry powder blend is compacted under modest forces and remilled to afford granules of specified particle size. The granules are then mixed with magnesium stearate and tabletted as stated above.

### EXAMPLE 2

Intravenous Formulations

Intravenous formulations of a selective carbonic anhydrase isoform IX inhibitor identified according to the procedure described above are prepared according to general intravenous formulation procedures known in the art, using D-glucuronic acid, mannitol NF, 1 N sodium hydroxide, and water. Various other buffer acids, such as L-lactic acid, acetic acid, citric acid or any pharmaceutically acceptable acid/conjugate base with reasonable buffering capacity in the pH range acceptable for intravenous administration may be substituted for glucuronic acid.

### EXAMPLE 3

Eye Drops

Solution compositions for topical administration containing Active I are prepared as illustrated below:

| | | |
|---|---|---|
| Active I | 6400 | mg |
| 0.5% hydroxyethylcellulose | 1 | L |

Active I is dissolved directly into 0.5% hydroxyethylcellulose to form a solution. The formulation is rendered sterile by starting the preparation procedure with sterile components and proceeding under sterile conditions.

### EXAMPLE 4

Eye drops

Additional eyedrop formulations are prepared having the following composition:

| | |
|---|---|
| Active I | 0.5% |
| Benzalkonium chloride solution | 0.02% v/v |
| Disodium edetate | 0.05% |
| NaCl | 0.8% |
| Water | to 100% |

### EXAMPLE 5

Ophthalmic Inserts

Ophthalmic inserts are manufactured from compression molded films which are prepared on a Carver Press by subjecting the powdered mixture of 1 mg Active I and 12 mg hydroxymethylcellulose to a compression force of 12,000 lbs. (gauge) at 300 degrees F. for one to four minutes. The film is cooled under pressure by having cold water circulate in the platen. Ophthalmic inserts are then individually cut from the film with a rod-shaped punch. Each insert is placed into a vial, which is then placed in a humidity cabinet (88% R.H. at 30 degrees C.) for two or four days. After removal from the humidity cabinet, the vials are stoppered from the humidity cabinet, the vials are stoppered and then capped. The vials containing the hydrate insert are then autoclaved at 250 degrees F. for one-half hour.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the instant invention as indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims that follow and that such claims be interpreted as broadly as is reasonable.

What is claimed is:

1. A composition for treating a macular condition selected from the group consisting of macular degeneration and macular edema, comprising a therapeutically effective amount of a carbonic anhydrase isoform IX inhibitor and a pharmaceutically acceptable carrier.

2. The composition of claim 1, wherein the composition is an ophthalmic preparation.

3. The composition of claim 2, wherein the composition comprises a solution, gel, semisolid, suspension, metered dose device, transdermal patch or film.

4. The composition of claim 2, wherein said ophthalmic preparation comprises a carbonic anhydrase isoform IX inhibitor at a concentration of about 0.01% weight/volume to about 5% weight/volume.

5. A method for treating a macular condition selected from the group consisting of macular degeneration and macular edema in a patient, by administering to the patient a therapeutically effective amount of a carbonic anhydrase isoform IX inhibitor.

6. A method of claim 5, wherein the macular condition results from retinitis pigmentosa or diabetic retinopathy.

7. A method of claim 5, wherein the macular degeneration is age-related macular degeneration or an inherited form of macular degeneration.

8. A method of claim 7, wherein the inherited form of macular degeneration is selected from the group consisting of Best's disease and Stargardt's macular dystrophy.
